(19) United States
(12) Patent Application Publication
(10) Pub. No.: US 2014/0073011 A1
(43) Pub. Date: Mar. 13, 2014

(54) METHOD FOR THE PRODUCTION OF AQUEOUS NUTRIENT SOURCE FOR ALGAE AQUACULTURE FARMING

(71) Applicants: Oyvind BRANDVOLL, Oslo (NO); Harald JOHANSEN, Sorumsand (NO); Ingo MACHENBACH, Oslo (NO); Arne RAAHEIM, Nannestad (NO)

(72) Inventors: Oyvind BRANDVOLL, Oslo (NO); Harald JOHANSEN, Sorumsand (NO); Ingo MACHENBACH, Oslo (NO); Arne RAAHEIM, Nannestad (NO)

(21) Appl. No.: 14/051,645

(22) Filed: Oct. 11, 2013

Related U.S. Application Data
(63) Continuation-in-part of application No. PCT/NO2012/050062, filed on Apr. 11, 2012.

(30) Foreign Application Priority Data
Apr. 11, 2011 (NO) 20110553
Jul. 1, 2011 (US) 61503645
Oct. 11, 2012 (NO) PCT/NO12/00058

(51) Int. Cl. C12N 1/12

(52) U.S. Cl. CPC C12N 1/12
USPC 435/71.1; 435/257.1; 435/257.6; 435/257.3; 435/41; 435/183

(57) ABSTRACT

Method for the production of aqueous nutrient source for macro and micro algae aquaculture farming and nutrient source thus formed. Phosphorous containing rocks are dissolved in at least one mineral acid to yield a solution containing a blend of ions of the minerals included in the rocks and in the acid in a form digestible by the algae and adding carbon in the form of CO₂, carbonic acid, or carbonate salts. The invention is also directed to a method for feeding an algae aquaculture farm with such nutrient source.

This application is a continuation-in-part application of PCT/NO2012/050062, filed Apr. 11, 2012, and claims priority to PCT/NO2012/000058, filed Oct. 11, 2012, U.S. application No. 61/503,645, filed Jul. 1, 2011, and Norwegian application number 20110553, filed Apr. 11, 2011, the contents of which are incorporated herein by reference.

The present invention gives a method for production of a nutrient source for algae aquaculture farming. According to a second aspect it concerns a nutrient blend produced by said method and according to a third embodiment it concerns a method for feeding an algae aquaculture farm.

### BACKGROUND

Microalgae, and other phototrophic microorganisms, are a diverse group of microorganisms that reside in marine and freshwater habitats. The key process for microalgae growth is photosynthesis, a process that uses light energy to convert CO₂, water and nutrients into hydrocarbons, while oxygen is being discharged.

Ranged at the bottom of the food chain, microalgae are capable of synthesizing essential products for higher living organisms, e.g. carotenoids, antioxidants, fatty acids, peptides, and enzymes. Omega fatty acids, for instance, are extracted from fish oil in large quantities and sold as dietary supplements due to their alleged positive effects on human health. Ackman et al. (1964) reported that fish do not synthesize long chain omega-3 fatty acids in significant quantities but acquire them through their diet by eating zooplankton that have fed on microalgae.

Microalgae have been investigated as energy source for several decades with the US DOE's Aquatic Species Program (1978-1996) and the Japanese RITE Program (1990-2000) being the most prominent research investments. The driving force behind most of this research is the high biomass yield from microalgae compared to terrestrial feedstock. Biomass yield from microalgae ranges 7-31 times higher than oil palm, the best oil yielding terrestrial plant (Kent and Andrews, 2007). Furthermore, their naturally high lipid content (20-50% of dry weight) renders cultivation of microalgae interesting as source for biofuel production. However, bio-energy production from algae culture has not gained a competitive edge so far, simply because the production cost at present is too high. Thus, research on microalgae, to bring the production costs down, are now receiving considerable attention. This research incentive is enhanced in view of the CO₂ mitigation question, where a future large industrial scale microalgae production could play an important part in “Carbon Capture and Storage” (CCS).

Photosynthetic growth requires light, carbon dioxide, water and nutrients. Algae growth in marine and freshwater bodies is generally limited by the availability of nutrients. Therefore, uncontrolled discharges of sewage or agricultural run-off can trigger algae blooms, also called eutrophication.

In production facilities, microalgae are grown in open ponds or in photo bioreactors, allowing for controlled and optimized growth conditions. Photobioreactor technology systems are however often associated with high capital and operational costs. The area efficiency also needs to be improved. These are all issues that must be significantly improved in order to meet the cost reduction requirements in future biofuel plants based on algae.

CO₂-binding capacity and sources. Micro-algal biomass contains approximately 50% carbon by dry weight (Christi, 2007) depending on the species. Since phototrophic algae species use CO₂ as their sole carbon source, production of 100 tons of algal biomass fixes roughly 183 tons of carbon dioxide. Most microalgae can tolerate high levels of CO₂, typically up to 150 000 ppmv (Bilanovic et al., 2009). However, the pH-decreasing effect of CO₂ dissolution must be buffered as most species achieve highest productivity at neutral pH.

Nutrients. Microalgae are grown in an aqueous growth medium that provides the inorganic elements constituting the algal cell. Carbon, nitrogen and phosphorous are the most important nutrients (macronutrients) for algae cultivation. Diatom algae require silica as macronutrient to build their outer cell walls. Other (potentially) important nutrients include calcium, magnesium, sodium, potassium, and sulphur. Micronutrients, trace elements required by plants and animals in very small quantities, include manganese, copper, zinc, cobalt, and molybdenum (Horn and Goldman 1994). The growth medium must contain nutrients in significant excess. For instance, phosphorus—normally added as phosphate—forms complexes with metal ions, rendering part of the added phosphorous unavailable to uptake by algae cells. While fast-growing species prefer ammonium over nitrate as nitrogen source, some microalgae can fix nitrogen in the form of NOₓ (Brennan and Owende, 2010). In order to minimize nutrient consumption, the growth medium must be recycled efficiently and nutrients consumed by algae growth must be replenished.

Today, profitable production of microalgae raw material used in refined products of the medicine, pharmacy, dietary supplement and cosmetics industries, is very profitable. Raw materials used as ingredients in fish and animal feeds may also in some cases be profitable.

However, large scale profitable industrial algae production for the energy or (bio) fuel markets, has currently several challenges, including; scale-up of the photobioreactor systems, energy consumption, aerial production efficiency, biomass harvesting, as well as supply of water, CO₂, and nutrients.

Different types of wastewaters may be used for cost efficient utilization of nutrients (particularly phosphorous and nitrogen) in industrial scale algae production in both photobioreactors and/or open ponds. However, regardless of the production method, nutrients from any type of wastewater may contaminate algae products, including possible costly contamination of algae production plants (leading to shut downs), and would thus be of limited use. In addition, open ponds have limited usage regardless of nutrient source, where algae produced in this manner may be satisfactory used in for example biofuel, while the use (because of contamination from air) in any type of food or medicine production, is not. The need for future cost efficient methods for obtaining “clean” nutrients, at any scale of algae production plants, is therefore desirable. Systems for production of biofuels with algae cultures are known e.g. from EP 2430 175, US 2012 053355, and from WO2011063129, which are incorporated herein by reference.

When algae are to be produced in large quantities (for example for biofuel), considerable amounts of nutrients are needed. This is illustrated in the following case for a 1 million tons/annum (Mt/a) CO₂ source.

Actual CO₂ fixation ratios per mass unit biomass vary with the algae species. However, assuming a strictly phototrophic species, a CO₂ fixation ratio of

f=1.83 g_{CO2}/g_{biomass}

can be expected (Christi, 2007).

An algae plant of this size would yield about 550 000 tons of dry algae mass per year. Minimal nutrient consumption estimated based on an average elemental algae composition determined by ECN (www.ecn.nl/phyllis) yields:

TABLE 1
Annual nutrient consumption for binding 1 Mt CO₂ in algae.

| | | Algae composition | Consumption* |
|---|---|---|---|
| Nutrient | | (% dry wt) | (in 1000 t) |
| Nitrogen | N | 8.01 | 44.1 |
| Potassium | K | 0.64 | 3.5 |
| Sulphur | S | 0.49 | 2.7 |
| Phosphorous | P | 0.27 | 1.5 |
| Magnesium | Mg | 0.14 | 0.8 |
| Sodium | Na | 0.10 | 0.5 |
| Calcium | Ca | 0.07 | 0.4 |
| Silicon | Si | 0.01 | 0.1 |

*It should be noted that these value are established on a purely theoretical basis and shall only serve to predict orders of magnitude.

Table 1 shows that the annual consumption of nutrients for binding 1 Mt CO₂ in algae is substantial. Phosphorous for example, is not found as free element in nature and is considered a limited, not-renewable resource. Closing phosphorous cycles is expected to become one of the major challenges in the future. The estimated phosphorous consumption of 1 400 tₚ/a is significant in view of the total estimated 2009-consumption of 8 000 tₚ/a in Norwegian agriculture. Using primary sources of phosphorous for algae cultivation, would therefore not be sustainable and compete with crop production. Large-scale algae culture is dependent on a sustainable source of phosphorous and efficient recycling of the growth medium.

The phosphorous load currently collected as sewage sludge and organic waste in

Norway is estimated to about 5 000 tₚ/a. A large fraction is readily recycled into agriculture in the form of stabilized sludge. Phosphorous-rich waste streams from human consumption frequently contain significant amounts of nitrogen and other inorganic compounds that could be used by algae. However, such waste streams also contain large amounts of carbonaceous material, unwanted inorganic species (e.g. heavy metals) and microorganisms that can interfere with algae growth.

Phosphorous taken directly from natural rocks would on the other hand not compete with food production. Such a phosphorous source would neither introduce the same problems as phosphorous taken from different types of waste streams.

### OBJECTIVES

Considering prior art technology it is an objective of the present invention to provide a method allowing cost-effective, industrial scale production of necessary nutrients for macro and microalgae growth in aquatic solutions.

It is a still further object to provide a process as mentioned above to introduce CO₂ (or carbonic acid) to the remaining liquid, which now contain all the necessary ingredients for clean and efficient algae growth.

It is a still further object to provide a process as mentioned above, which is principally sustainable in all its aspects, allowing sustainable algae farming production of a number of products.

### SUMMARY OF THE INVENTION

The above objects are achieved by the present invention which according to a first embodiment concerns a method.

According to a second aspect the present invention concerns a nutrient blend for macro- and microalgae aquaculture farming.

According to a third embodiment the present invention concerns a method of feeding an algae aquaculture farm with the nutrient blend provided.

Finally the present invention concerns a method for producing chemical products.

Preferred and optional embodiments of the invention are disclosed by the dependent claims.

As used herein “algae aquaculture farm” and “algae aquaculture farming” refers to any arrangement in which algae is allowed to grow in aqueous medium under humanly controlled conditions, be it in a bioreactor, in open ponds or in any other suitable arrangement.

As used herein “microalgae” refers to microalgae and other phototrophic microorganisms.The present invention provides a method for production of necessary clean nutrients, especially in the form of phosphorous for macro- and microalgae growth in aquatic solutions from natural rocks, mine tailings, and other rock wastage (for example apatite containing plagifoyaite/nepheline syenite). The rocks/rock-mix or rock products are dissolved, or partly dissolved, in mineral acids (e.g. HNO₃, H₂SO4, HCl). Undissolved material and precipitated, amorphous SiO₂are separated from the liquid an may serve as raw material for example for elements such as Si, Fe, Ti, Zr, U, Th and rare earth elements, depending on rock-types used. The material in solution is diluted with water and when diluted, ammonia (if needed) may be added. NaOH and/or KOH may also be added, mainly for pH adjustment, at this stage, Precipitates, mainly Al-hydroxide (and some Fe-hydroxide) are separated from the liquid, and may be used as raw material for different industrial products.

CO₂, carbonic acid, or carbonate salts, for example extracted from carbonate rich rocks such as the carbonatite rock, sovite (see FIG. 5), are then introduced to the remaining liquid, which now contains all the necessary ingredients for clean and efficient algae growth. By varying the rock types or mixture of rocks, nutrients may be tailor-made for different algae species.

While growth of macroalgae are mostly limited to open ponds and other open algae growth environments, microalgae allow more versatile production facilities than macroalgae and are therefore preferred. Non-limiting examples of preferred microalgae are Chlamydomonas reinhardtii, Chlorella vulgaris, Spirulina platensis, Haemato 'coccus pluvialis, Botryococcus braunii, Nannochloropsis occulata, Crypthecodinium cohnii, or Thraustochytrium roseum.

A major aspect of the present invention is the sustainability achieved by allowing very large scale production of e.g. biofuels in a manner not competing with food production, by directly extracting the valuable phosphorous from rocks.

Seawater (or brines/saline aquifers adjusted by e.g. nanofiltration) may be used instead of fresh water. Since seawater contains desirable amounts of salts and ions, such as SO₄²⁻ and Cl⁻, seawater is preferred over freshwater in many areas of use of the present invention. The need for addition or use of sulphuric acid and hydrochloric acid is thereby eliminated, opening for use of the more preferred nitric acid which in itself is an important source of nitrogen.

It is also an option to dissolve, or partly dissolve suitable rock-types or mixture of rocks in carbonic acid and adjust the resulting liquid, by mixing in, lacking ingredients from rocks of suitable composition dissolved in nitric acid, or other mineral acid, to achieve an optimal nutrient composition tailor-made for the growth of different algae species. A variant of this process is to achieve carbon nutrients from carbonate salts, directly form crushed carbonate rocks in seawater slurry, with CO₂ only from air, and adjust the resulting liquid, by mixing in, lacking ingredients from rocks of suitable composition dissolved in nitric acid, or other mineral acid, to achieve an optimal nutrient composition tailor-made for the growth of different algae species, as illustrated in FIG. 6.

Silicon containing minerals (silicates) suitable for the present process are formed because of insufficient content of SiO₂ in the original magma (rock melt), where examples of such minerals are; nepheline (Na, K)(AlSiO₄), leucite K(AlSi₂O₆) (occurring in volcanic rocks), and olivine Mg₂(SiO₄). The largest known mass of nepheline rocks is found on the Kola Peninsula in Russia, where such rocks are associated with large amounts of apatite (a major phosphorous source for the fertilizer-industry). Extensive masses of nepheline rocks are also found in the Oslo region of Norway.

TABLE 2
Examples of chemical analyses of plagifoyaite (local name;
lardalite) and nepheline syenite (local name foyaite/hedrumite)
are taken from E. R. Neumann (1977).

Local name

| Streckeisen | Lardalite | Foyaite/hedrumite |
|---|---|---|
| classification | Plagifoyaite | Nepheline syenite |
| SiO₂ | 55.55 | 55.90 |
| TiO₂ | 1.12 | 0.61 |
| Al₂O₃ | 20.38 | 23.18 |
| Fe₂O₃ | 2.06 | 1.30 |
| FeO | 2.42 | 0.67 |
| MnO | 0.16 | 0.09 |
| MgO | 1.46 | 0.44 |
| CaO | 2.32 | 1.03 |
| Na₂O | 8.15 | 10.57 |
| K₂O | 5.04 | 5.70 |
| P₂O₅ | 0.64 | 0.08 |
| H₂O | 0.75 | 0.58 |
| Sum | 100.5 | 100.15 |

In addition to the elements (given as oxides) in the table above, such rocks usually contain the necessary micro-nutrients, or trace elements, necessary for algae growth.

If, on the other hand, suitable rocks do not contain enough macro-nutrients, a rock-mix, containing all macro- and micro-nutrients, can readily be made. For example if the present rock in question does not contain enough phosphorous, sovite, a carbonatite rock from the Fen-area of Telemark, Norway, can be mixed with other suitable rocks, as sovite contain up to 30% apatite.

When such a rock, or rock mix, is dissolved in nitric acid and a suitable portion (according to the growth requirements of different algae species) is dissolved in sulphuric acid (to achieve the necessary macro-nutrient sulphur) together with a portion in hydrochloric acid, if chlorine ions are needed, (see FIG. 1) most of the important nutrients for algae growth, would now be available. When undissolved and precipitated materials from the acid dissolution step are removed, the remaining acidic liquid is diluted with water and if needed for efficient algae growth ammonia and/ or ammonium salts may be added. Ammonium ions may be preferred before nitrates as nitrogen containing nutrient.

Addition of CO₂ to this liquid gives a complete nutrient mix in the necessary amounts for optimal algae growth, where also the nutrient mix can be tailor-made for different algae species. It should be noted that nutrients from silicate rocks will also include silica for silicic algae species (diatom algae).

In order to optimize nutrient consumption, the growth medium is recycled efficiently and nutrients consumed by algae are compensated.

This compensation can be performed by “bleeding in” necessary amounts of nutrients, with natural rocks, mineral acids, water, optionally ammonia, and CO₂, as primary sources. NaOH or KOH may be added as a pH adjusting agent.

Undissolved and precipitated (mainly amorphous silica) materials from the acid dissolution step are separated from the liquid and may serve as raw material for example for elements such as; Si, Fe, Ti, Zr, Th, U and rare earths, depending on rock types used in processes not described in detail here.

Precipitated material, in the water dilution step, mainly Al-hydroxides (and some Fe-hydroxides, depending on rock type), may be used as raw material for different industrial products, including alumina.

In one embodiment of the present invention seawater is used in the dilution step. Since seawater contains necessary amounts of sulphur (and chlorine) nitric acid may be used as the sole acid in the dissolution step.

Another variant of the invention is to dissolve suitable rock types/rock-mix in carbonic acid (CO₂ and water) and adjust the resulting liquid by bleeding in, or mixing in, lacking nutrient ingredients from rocks of suitable composition dissolved in nitric acid (or other mineral acids, if needed), to achieve an optimal nutrient composition, tailor-made for growth of different algae species.

It is also an option to adjust the solvent after the carbonic acid treatment, by using seawater at the “bleeding in stage” of lacking nutrients.

In both the latter variants of the invention, undissolved and precipitated material, from the carbonic acid dissolution process, may contain raw material for economically interesting elements.

### BRIEF DESCRIPTION OF THE DRAWINGS

FIG. 1 is a schematic flow diagram of a first embodiment of the process according to the present invention.

FIG. 2 is a schematic flow diagram of a second embodiment of the process according to the present invention.

FIG. 3 is a schematic flow diagram of a third embodiment of the process according to the present invention.

FIG. 4 is a schematic flow diagram of a fourth embodiment of the process according to the present invention.

FIG. 5 is a schematic flow diagram of a fifth embodiment of the process according to the present invention

FIG. 6 is a schematic flow diagram of a sixth embodiment of the process according to the present invention.

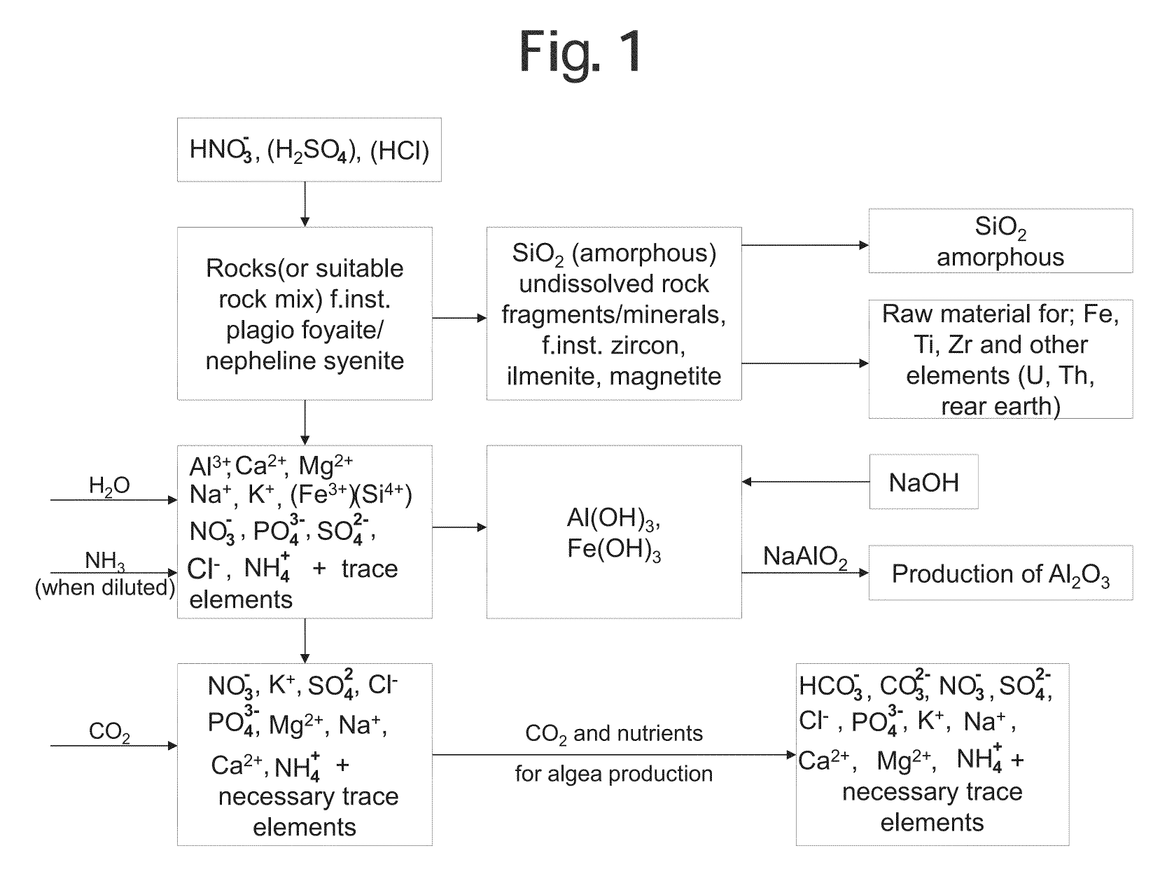

FIG. 1 describes a version of the present invention where rocks, or a suitable composition of rocks, are dissolved in mineral acids (e.g. nitric acid, sulphuric acid, hydrochloric acid). Amorphous silica and undissolved rock fragments/minerals are separated from the solvent, and may serve as raw material for silica, and other valuable elements and compounds.

The solvent from the dissolving process is diluted with water and when diluted to concentrations acceptable for algae growth ammonia may be added, if the nitrogen nutrient in the form of nitrate ions, is not considered sufficient. Precipitates from this process are separated from the liquid, and may be used for different purposes.

CO₂, carbonic acid, or carbonate salts are then introduced to the remaining liquid, which now contains all the necessary ingredients for clean and efficient algae growth.

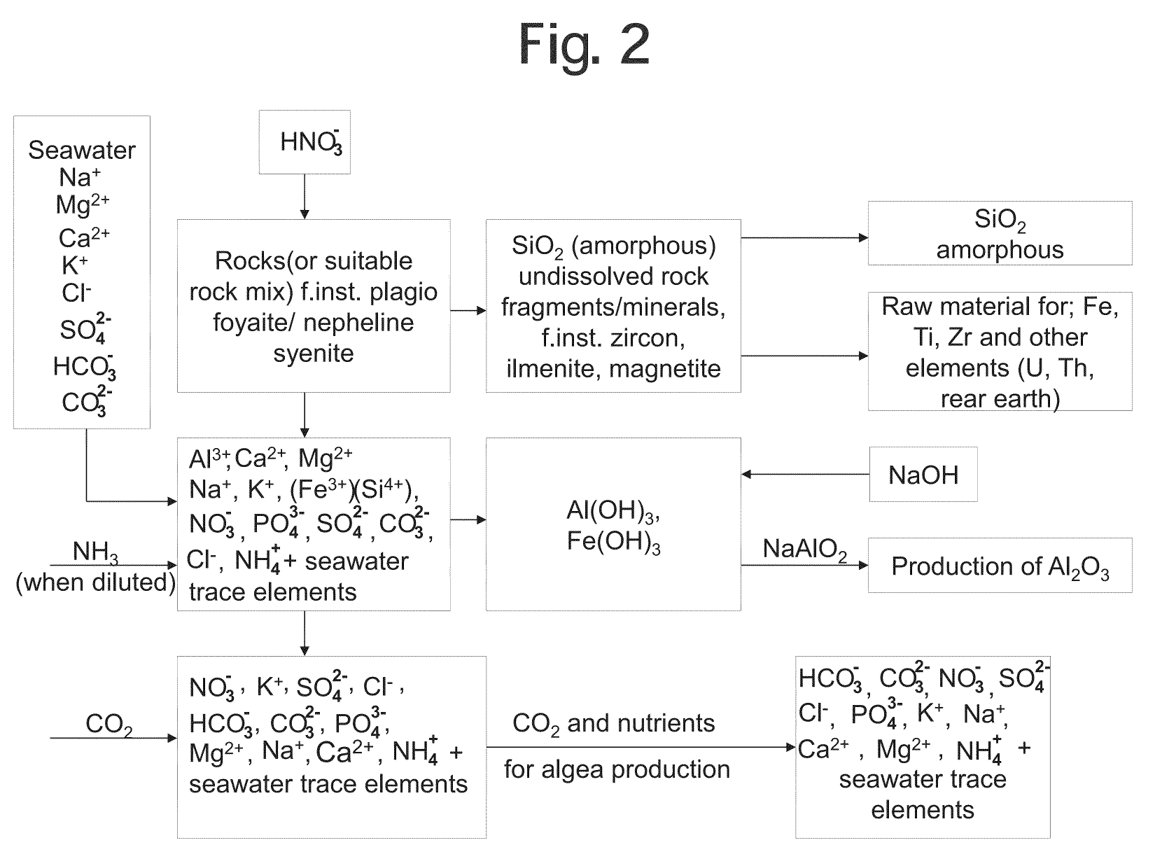

FIG. 2 describes a process very similar to that given in FIG. 1, except that seawater is used instead of fresh water. As seawater contain sufficient amounts of sulphate and chlorine ions, the use of nitric acid as the sole acid in the dissolution process of natural rocks, would be satisfactory. Brines (or chemically adjusted (by(nano)-membranes) brines/saline aquifers) may also be used.

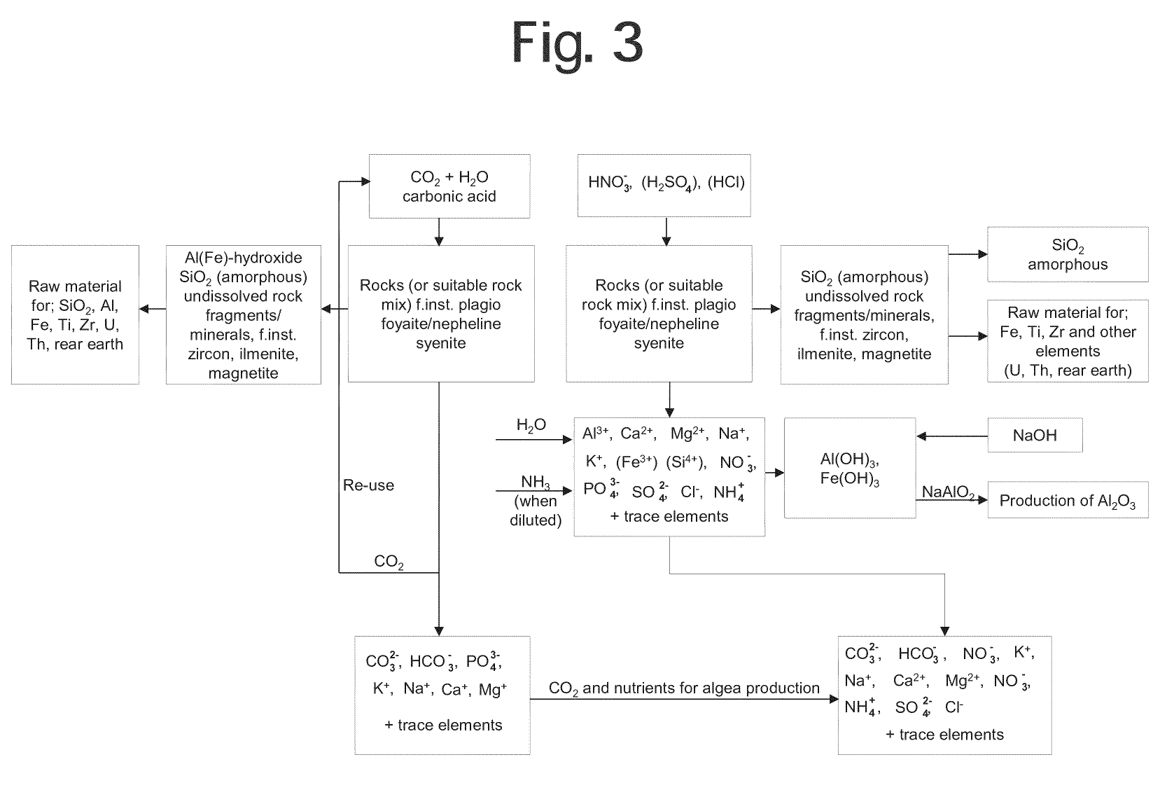

FIG. 3 shows a third embodiment of the present method, in which is included an option to dissolve, or partly dissolved, suitable rock-types or mixture of rocks in carbonic acid and adjust the resulting liquid, by mixing in, lacking ingredients from rocks of suitable composition dissolved in nitric acid, or other mineral acid, to achieve an optimal nutrient composition tailor-made for the growth of different algae species.

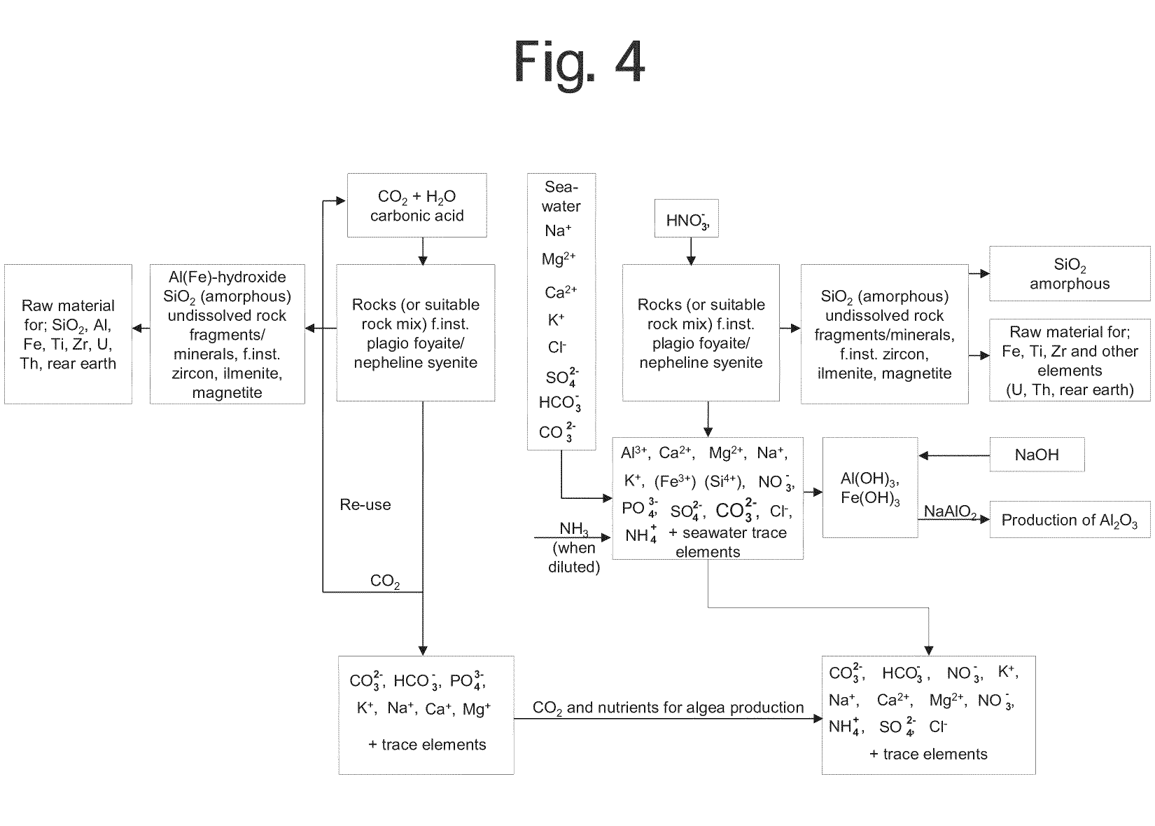

FIG. 4 describes a process very similar to that given in FIG. 3, except that seawater is used instead of fresh water in the dilution step of the acidic liquid, from the acid dissolution step.

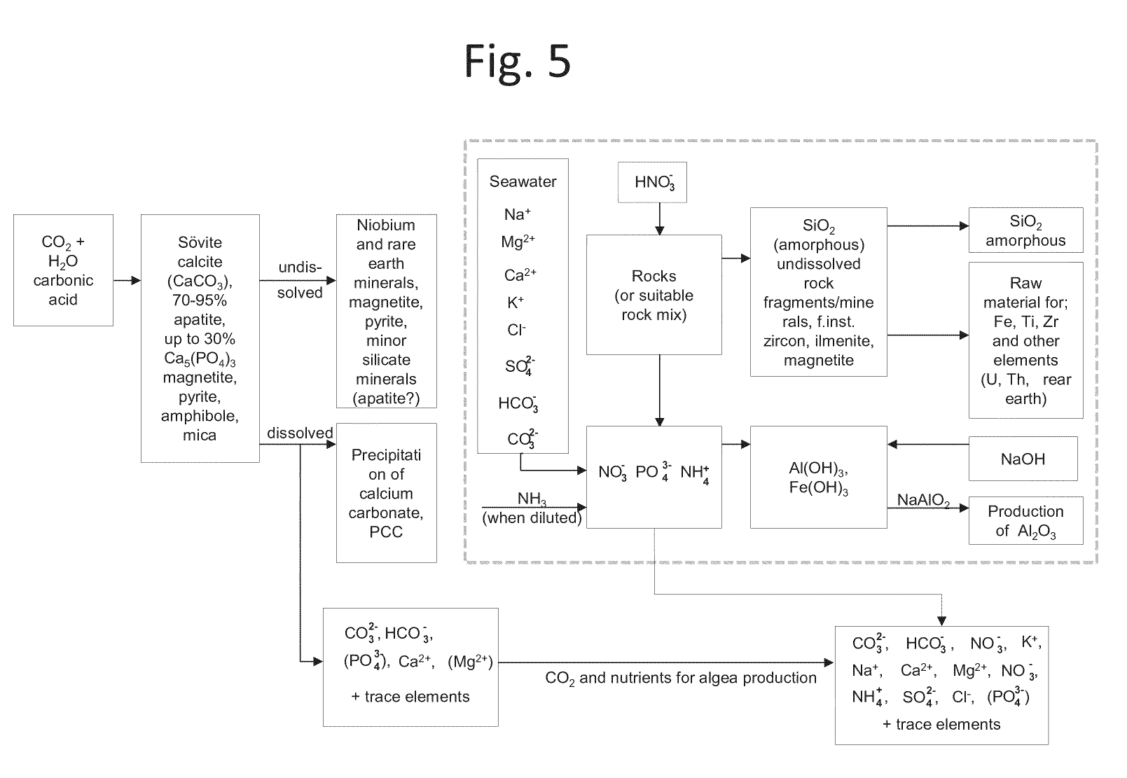

FIG. 5 describes a process very similar to that given in FIGS. 3 and 4, except that the rock used for carbonic acid dissolution is a carbonate containing rock, exemplified by sovite.

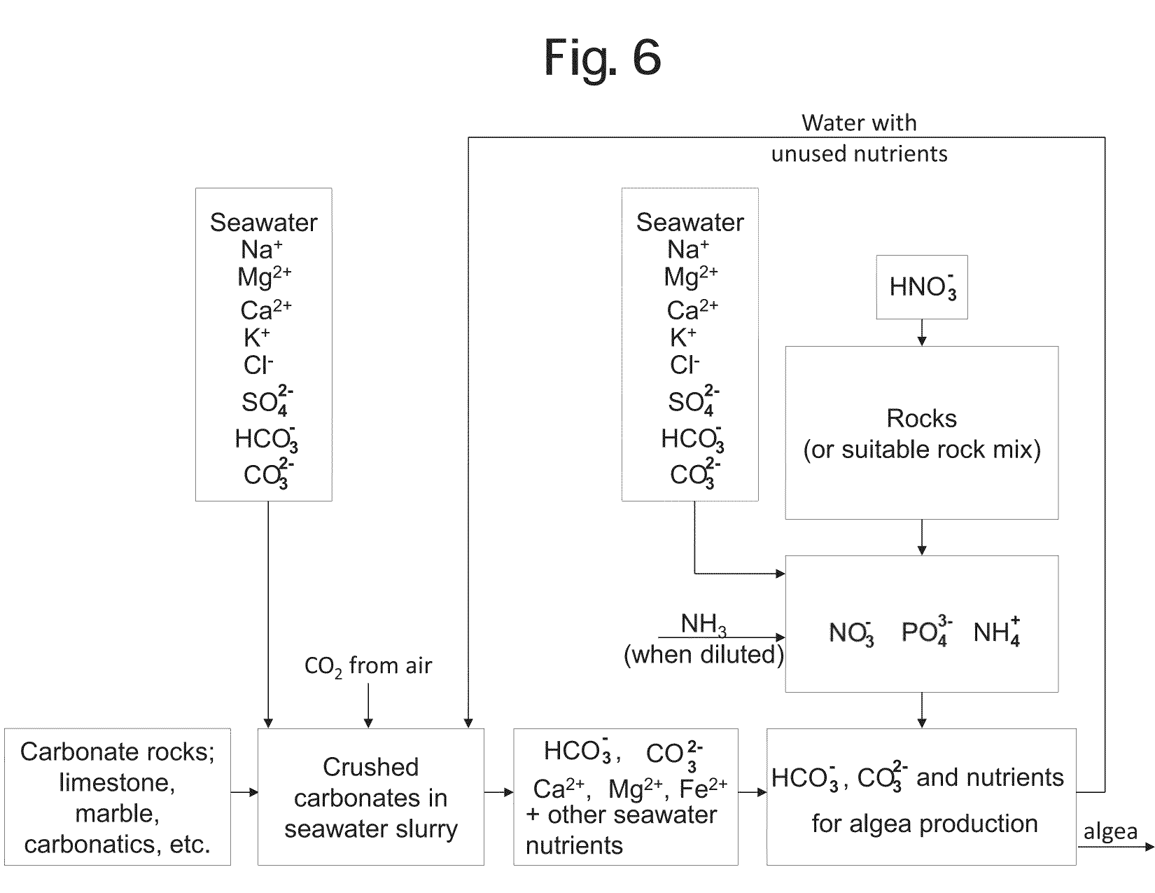

FIG. 6 describes a process where carbon nutrients is obtained from carbonate salts, directly form crushed carbonate rocks in seawater slurry, with CO₂ only from air. The resulting liquid is adjusted, by mixing in, lacking ingredients from rocks of suitable composition dissolved in nitric acid, or other mineral acid, to achieve an optimal nutrient composition tailor-made for the growth of different algae species.

In the method according to the present invention the naturally occurring rocks are selected as to contain a convenient mixture of minerals in relation to the algae nutrition needs, which may differ from algae specie to algae specie. The naturally occurring rocks may include apatite containing rocks typically selected among plagifoyaite, nepheline-syenite and carbonatite, or any combination of same. Typically a combination of rock types, compositions and acid(s) for their dissolving are selected so as to tailor the blend of minerals for a particular type of algae.

The mineral ion solution comprising mineral acid may prior to its use as nutrient be combined with a mineral ion solution resulting from rocks being dissolved by carbonic acid.

It should be noted that it is not within the object or context of this invention to establish which mineral blend is optimal for any particular algae specie. Neither is it within the object of this invention to establish the optimal growth conditions for the algae, with regard to pH or other controllable parameters.

The nutrient blend produced is used to feed algae in an aquaculture plant designed for production of products selected among carotenoids, antioxidants, peptides, enzymes, ingredients in fish and animal feeds, biomass feedstock for energy conversion, and biofuels. It should be noted that algae farming allows such production already, but not in a sustainable manner when scaled up to industrial scale.

The nutrient blend is preferably fed continuously to the algae aquaculture farm in a loop allowing recycling of unused nutrients while bleeding in fresh nutrients at a rate of which nutrients are consumed.

### REFERENCES

Ackman R. G., Jangaard P. M., Hoyle R. J. and Brockerhoff H. (1964). Origin of marine fatty acids. I. Analysis of the fatty acids produced by the diatom Skeletonema costatum. J. Fish. Res. Board Canada 21, 747-756.

Bilanovic D., Andargatchew A., Kroeger T. and Shelef G. (2009). Freshwater and marine microalgae sequestering of CO₂ at different C and N concentrations—response surface methodology analysis. Energy Cony. Managem. 50(2), 262-267.

Christi Y. (2007). Biodiesel from microalgae. Biotechnol. Adv. 25, 294-306.

Horne A. J. and Goldman C. R. (1994). Limnology Second Edition. McGraw Hill Inc., New York, USA.

Kent M. S. and Andrews K. M. (2007). Biological research survey for the efficient conversion of biomass to bio-fuels. Sandia report SAND2006-7221, Sandia National Laboratories, USA.

Else-Ragnhild Neumann: Petrology of the plutonic rocks, in Paleorift systems with emphasis on the Permian Oslo rift (1977): A review and guide to excursions/Ed. Johannes A. Dons; NGU-series; 337, published 1978.

We claim:

1. A method for the production of aqueous nutrient source for algae aquaculture farming, wherein in that phosphorous containing rocks are dissolved in at least one mineral acid to yield a solution containing a blend of ions of the minerals included in the rocks and in the acid in a form digestible by the algae and adding carbon in the form of CO₂, carbonic acid, or carbonate salts.

2. A method as claimed in claim 1, wherein the mineral solution prior to its use as nutrient is diluted with water.

3. A method as claimed in claim 2, wherein the water is selected among fresh water, seawater, brine or saline aquifer or any combination of two or more of same.

4. A method as claimed in claim 3, wherein the water is chosen among water containing SO₄²⁻ ions and Cl⁻ ions.

5. A method as claimed in claim 1, wherein ammonia is added to the mineral solution prior to its use as nutrient.

6. A method as claimed in claim 1, wherein CO₂ is added to the mineral solution prior to its use as nutrient.

7. A method as claimed in claim 1, wherein the rocks are naturally occurring rocks from mine tailings or rock wastage from other rock recovery processes.

8. A method as claimed in claim 1, wherein the mineral acid is selected from the group consisting of HNO₃, H₂SO₄, HCl, and combinations of two or more thereof.

9. A method as claimed in claim 1, wherein the mineral acid is HNO₃.

10. A method as claimed in claim 1, wherein the naturally occurring rocks include apatite containing rocks.

11. A method as claimed in claim 10, wherein the apatite containing rocks are selected from the group consisting of plagifoyaite, nepheline-syenite, carbonatite, and combinations thereof.

12. A method as claimed in claim 1, wherein the mineral ion solution comprising mineral acid, prior to its use as nutrient is combined with a mineral ion solution resulting from rocks being dissolved by carbonic acid.

13. A method as claimed in claim 7 , wherein materials precipitating out of solution are recovered for optional use in production of side-products.

14. A method as claimed in claim 1, wherein a combination of rock types, compositions and acid for their dissolving are selected so as to tailor the blend of minerals for a particular type of algae.

15. A method as claimed in claim in claim 1, wherein the nutrient blend produced is used to feed algae in an aquaculture plant designed for production of products selected from the group consisting of carotenoids, antioxidants, peptides, enzymes, ingredients in fish and animal feeds, biomass feedstock for energy conversion, and biofuels.

16. A method as claimed in claim 15, wherein the production is industrial scale production.

17. A method as claimed in claim 1, wherein the algae are microalgae.

18. A method as claimed in claim 17, wherein the microalgae species are selected from the group consisting of Chlamydomonas reinhardtii, Chlorella vulgaris, Spirulina platensis, Haemato 'coccus pluvialis, Botryococcus braunii, Nannochloropsis occulata, Crypthecodinium cohnii, and Thraustochytrium roseum.

19. A method in accordance with claim 1, wherein a bioreactor is used to carry out the method.

20. A nutrient blend for macro- and microalgae aquaculture farming produced by the method according to claim 1.

21. A method for feeding an algae aquaculture farm, wherein a nutrient blend according to claim 20, is fed to the algae aquaculture farm subsequent an optional pH adjustment.

22. A method according to claim 21, wherein the nutrient blend is fed continuously to the algae aquaculture farm in a loop allowing recycling of unused nutrients while bleeding in fresh nutrients at a rate of which nutrients are consumed.

23. A method for producing chemical products individually selected from the group consisting of carotenoids, antioxidants, peptides, enzymes, ingredients in fish and animal feeds, biomass feedstock for energy conversion, and biofuels by use of algae aquaculture farming, wherein the nutritional feed for the algae is provided by dissolving rocks in at least one mineral acid to yield a solution containing a blend of ions of the minerals included in the rocks and in the acid, in a form digestible by the algae, and adding carbon in the form of CO₂, carbonic acid, and carbonate salts.
